(19) United States
(12) Patent Application Publication
(10) Pub. No.: US 2013/0171028 A1
(43) Pub. Date: Jul. 4, 2013

(54) ANALYTE MONITOR

(71) Applicant: BAYER HEALTHCARE LLC, Tarrytown, NY (US)

(72) Inventors: Gerald H. Shaffer, Elkhart, IN (US); Anurag Gupta, Tucson, AZ (US)

(73) Assignee: BAYER HEALTHCARE LLC, Tarrytown, NY (US)

(21) Appl. No.: 13/729,919

(22) Filed: Dec. 28, 2012

Related U.S. Application Data
(60) Provisional application No. 61/580,809, filed on Dec. 28, 2011.

(51) Int. Cl. G01N 21/75; G01N 33/49

(52) U.S. Cl. CPC G01N 21/75; G01N 33/49
USPC 422/82.09; 356/41

(57) ABSTRACT

An analyte meter for an analyte test strip includes a light source configured to emit a light having a wavelength substantially similar to the maximum absorption band of Hb, an optics assembly configured to direct the light emitted by the light source to a test strip, and a photodetector configured to quantitatively detect light emanating from the test strip and to generate a signal correlating to an analyte concentration in the test strip.

### CROSS-REFERENCE TO RELATED APPLICATIONS

The present application claims the benefit of the filing date of U.S. Provisional Patent Application No. 61/580,809 filed Dec. 28, 2011, the disclosure of which is hereby incorporated herein by reference.

### BACKGROUND OF THE INVENTION

The present disclosure generally relates to a body fluid analyte metering system and, more particularly, to hemoglobin A1c (HbA1c) metering systems.

Qualitative or semi-quantitative tests are generally appropriate for many analytes such as the markers for pregnancy and ovulation. Certain analytes, however, require accurate quantitation. For example, glucose, cholesterol, HDL cholesterol, triglyceride, a variety of therapeutic drugs such as theophylline, vitamin levels, and some other health indicators require precise quantitative tests.

Another specific analyte that requires accurate quantitation is hemoglobin A1c (HbA1c). HbA1c is a form of glycated hemoglobin that indicates a patient's blood sugar control over the preceding two to three month period and is formed when glucose in the blood combines irreversibly with hemoglobin to form stable glycated hemoglobin. Accordingly, HbA1c is eliminated only when the red blood cells are replaced. Since the normal life span of red blood cells is about 90 to 120 days, HbA1c values are directly proportional to the concentration of glucose in the blood over the full life span of the red blood cells and are not subject to fluctuations that are seen with daily blood glucose monitoring.

Measuring HbA1c is important because it may be used to evaluate the risk of health complications stemming from diabetes such as glycemic damage to tissues (e.g., nerves, and small blood vessels in the eyes and kidneys). Due to the importance of monitoring HbA1c levels, patients with diabetes mellitus now monitor their blood glucose levels themselves in home settings. These patients therefore need reliable devices and methods for quantitatively monitoring analytes such as HbA1c. Hence, a need exists for methods and devices capable of accurate quantitation of analytes such as HbA1C.

### SUMMARY OF THE INVENTION

The present disclosure relates to analyte meters for quantification of analytes such as HbA1c. In one embodiment, the analyte meter includes a housing, a light source in the housing that is configured to emit a light having a wavelength substantially similar to the maximum absorption band of Hb, an optics assembly configured to direct the light emitted by the light source to a test strip, and a photodetector configured to quantitatively detect light emanating from the test strip and to generate a signal correlating to an analyte concentration in the test strip. The light source may be configured to emit a green light having a wavelength ranging between 525 and 535 nm. Alternatively, the green light may have a wavelength of 530 nm.

In another embodiment, the analyte meter may further include a refracting element comprised of a plurality of lenses. The plurality of lenses may be arranged in an array and each of the lenses may be uniformly spaced apart from one another. There may be more than 10 lenses arranged in the array of microlenses, including a number between 10 and 250. There may also be more than 100 lenses in the array. A surface of at least one lens in the array of lenses may have a radius of curvature of about 100 μm, a conic constant (k) of −1, and maximum sag of about 56.25 μm. A pitch of the array of lenses may be 155 μm. Prior to the light reaching the test strip, the array of the plurality of lenses is the last surface area through which light travels.

In another embodiment, the analyte meter includes a housing, a first light source in the housing configured to emit a first light, a second light source configured to emit a second light, an assay strip including first and second reaction zones, a first photodetector, a second photodetector, and an optics assembly. The first reaction zone is adapted for receiving a fluid sample containing first and second analytes and includes a first reagent capable of inducing an optical change in the fluid sample when reacted with the first analyte. The second reaction zone is adapted for receiving the fluid sample and includes a second reagent capable of inducing an optical change in the fluid sample when reacted with the second analyte. The optics assembly is configured to direct the first light to the first reaction zone and the second light to the second reaction zone. The first photodetector is positioned so that it only detects optical radiation reflected from the first reaction zone and to generate a first signal indicative of an amount of analyte in the fluid sample positioned in the first reaction zone. The second photodetector is positioned so that it only detects optical radiation reflected from the second reaction zone and to generate a second signal indicative of an amount of analyte in the fluid sample positioned in the second reaction zone. In an alternative embodiment, the first and second analytes may be different analytes.

The optics assembly may further comprise a first refracting element positioned between the first reaction zone and the first photodetector. The first refracting element may have an optical axis extending through the first refractor in a direction between the first reaction zone and the first photodetector and the first photodetector may extend in a direction perpendicular to the optical axis. There may also be a second refracting element that is positioned between the second reaction zone and the second photodetector. The second refracting element may similarly have a second optical axis extending therethrough in a direction between the second reaction zone and the second photodetector. The second photodetector may also extend in a direction perpendicular to the second optical axis.

In an alternative embodiment, the optics assembly may further comprise a plurality of reflecting elements that directs light emanating from the first light source to the first reaction zone. The first light source may be aligned with at least one reflecting element.

In yet a further embodiment, an analyte meter for detecting analyte concentration in a test strip has a reaction zone adapted for receiving a fluid sample that contains an analyte. The meter further includes a reagent capable of inducing an optical change in the fluid sample when reacted with the fluid sample includes a light source configured to emit a light along an illumination path, an optics assembly configured to direct the light emitted by the light source to the reaction zone of the test strip, the optics assembly including an array of microlenses positioned along the illumination path, and a photodetector configured to quantitatively detect light reflected from the reaction zone and to generate a signal correlating to an amount of analyte in the fluid sample. The microlenses lenses in the array of microlenses may be uniformly spaced apart from one another.

The present disclosure further relates to an optics assembly for an analyte meter system. In some embodiments, the optics assembly includes a receiving portion adapted for receiving at least one test strip, the at least one test strip having a reaction zone adapted for receiving a fluid sample containing an analyte and including a reagent capable of inducing an optical change in the fluid sample when reacted with the analyte; a light source configured to emit a green light having a wavelength substantially similar to the maximum absorption band of Hb; at least one reflecting element configured to direct the light emitted by the light source to the reaction zone of a test strip in said receiving portion; and a photodetector configured to quantitatively detect light emanating from the reaction zone of the test strip and to generate a signal correlating to an amount of analyte in the fluid sample. The wavelength of the green light may range from 525-535 nm.

### BRIEF DESCRIPTION OF THE DRAWINGS

Various embodiments of the present invention will now be described with reference to the appended drawings. It is appreciated that these drawings depict only some embodiments of the invention and are therefore not to be considered limiting of its scope.

FIG. 1 is an exploded perspective view of an embodiment of an analyte meter or diagnostic device;

FIG. 2 is a perspective top view of an optics assembly of the diagnostic device of FIG. 1;

FIG. 3 is a perspective bottom view of the optics assembly of FIG. 2;

FIG. 4A is a top view of a refracting element including an array of microlens;

FIG. 4B is a side view of a microlens of the refracting element of FIG. 4A;

FIG. 5 is a schematic representation of the detection path of the optics assembly of FIG. 2;

FIG. 6 is a schematic representation of the illumination path of the optics assembly of FIG. 2; and

FIG. 7 is a graphic showing the spectral reflectance curves for the Hb sample pad.

### DETAILED DESCRIPTION

In the following are described the preferred embodiments of the analyte meter in accordance with the present invention. In describing the embodiments illustrated in the drawings, specific terminology has been used for the sake of clarity. However, the invention is not intended to be limited to the specific terms selected, and it is to be understood that each term includes all technical equivalents that operate in a similar manner to accomplish a similar purpose.

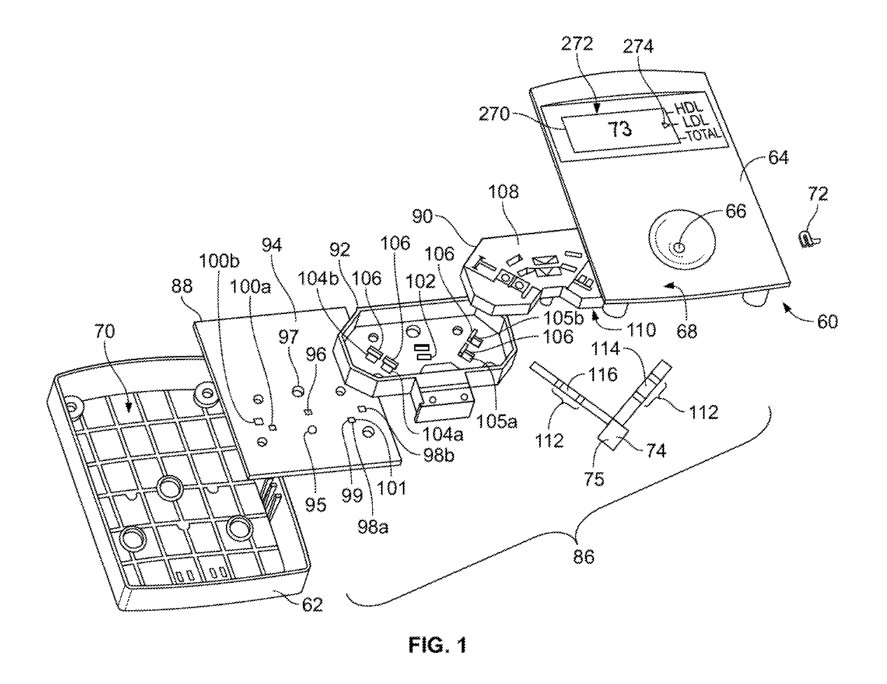

FIG. 1 illustrates an embodiment of an analyte meter or diagnostic device 60 for measuring HbA1c or other analytes. As used herein, the term “analyte” refers to the substance to be detected which may be present in the test sample, typically a body fluid. Suitable analytes include, but are not limited to, glucose, cholesterol, HDL cholesterol, LDL cholesterol, triglycerides, and BUN.

Meter 60 includes a housing 62 and cover 64 with a receptor, such as an inlet port 66. Inlet port 66 extends from the exterior surface 68 of the cover 64 to interior cavity 70 of the housing 62 and is dimensioned for receiving a sample 72 containing one or more selected analytes to be determined. Inlet port 66 allows the sample 72 to be introduced to a sample receiving device or receptor 74 positioned within interior cavity 70. Sample receiving device 74 includes a receiving pad 75 positioned in fluid communication with two assays strips 114 and 116 and serves to distribute the samples between the two strips. Typically, the receiving pad 75 is a two-layer pad. Optionally, sample receiving device 74 may include a sample filter pad for removing undesired contaminants from the sample. The sample filter pad may be the same as the receiving pad 75 with one pad performing both functions. Meter 60 may include more than one sample filter pad along the pathway of the sample flow for removing different types of contaminants.

The two assay strips 114 and 116 contain chemical reagents or any other suitable reagents for determining the presence of one or more selected analytes. In some embodiments, at least one assay strip 114 or 116 includes a reagent that reacts with a blood sample to yield a physically detectable change which correlates with the amount of selected analyte in the blood sample. In some embodiments, the reagents are capable of inducing an optical change in the fluid sample when reacted with the fluid sample. For instance, the reagent on each assay strip 114 or 116 may react with the blood sample so as to indicate the concentration of hemoglobin A1c (HbA1c). Examples of detection systems appropriate for use in measuring hemoglobin A1c are described in U.S. Pat. Nos. 5,837,564; 5,945,345; and 5,580,764, the disclosures of which are herein incorporated by reference in their entirety. It is understood, however, that the present disclosure is not limited to using such reagents and reactions. Other analytic possibilities are also contemplated.

The interior cavity 70 of the housing 62 encloses a reflectometer 86. Housing 62 may also enclose a desiccant and an absorptive material for controlling excess sample volume overflow. Reflectometer 86 includes a printed circuit board (PCB) 88, an optics assembly 90 and a shield 92. PCB 88 includes a processor (not shown) and has one top face 94 facing cover 64 when positioned within interior cavity 70 of housing 62. A reference detector 96 and zone detectors 98a, 98b, 100a, 100b are mounted directly on the face 94 of PCB 88. At least one of zone detectors 98a, 98b, 100a, 100b may be a photodetector or photosensor configured to quantitatively detect or sense light and generate an electrical signal correlating to such detected or sensed light. In other words, the photodetector or photosensor can convert an optical signal into an electrical signal. For example, the photodetector can quantitatively detect light emanating from an assay strip, 114 or 116, and generate an electrical signal. This electrical signal can be calibrated to correlate to an amount of analyte in the fluid sample on the assay strip.

The face 94 of PCB 88 also has at least two light sources 95, 97 suitable to emit light. Suitable light sources include light-emitting diodes (LEDs) and a light-emitting transistor (LET). Light sources 95 and 97 provide illumination in all directions above the face 94 of PCB 88. In the case where light sources 95 and 97 are LEDs, these LEDs may be in bare die form without an integral lens, enclosure, or housing. As a result, the LEDs provide illumination in all directions above the face 94 of the PCB 88. Similarly, zone detectors 98a, 98b, 100a, 100b and reference detector 96 may also be in bare die form mounted directly onto the face 94 of PCB 88. Light sources 95, 97 and detectors 96, 98a, 98b, 100a, 100b may be all positioned on the same plane.

In certain embodiments, at least one light source 95 or 97 emits a light having a wavelength identical or at least substantially similar to the maximum absorption band of hemoglobin (Hb), as discussed in further detail below. For example, light source 95 (or 97) may be an LED configured to emit a green light having a wavelength of 530 nm. For a given range of Hb concentration, this specific LED increases the dynamic range reflectance measurement from 6.7% to 8.4% over current designs, as discussed below. Light source 97 (or 95) may alternatively be configured to emit a red light.

A shield 92 is placed over the face 94 of PCB 88. The shield 92 has one or more apertures 102 aligned with light sources 95, 97, and reference detector 96. The shield 92 also has openings 104a, 104b, 105a, 105b, each aligned with one of the zone detectors 98a, 98b, 100a, and 100b. Apertures 102 prevent obstruction of light emitted from light sources 95, 97 or received by reference detector 96. Openings 104a, 104b, 105a, 105b allow light to reach zone detectors 98a, 98b, 100a, and 100b. Specifically, opening 104a is aligned with zone detector 100a. Opening 104b is aligned with zone detector 100b. Opening 105a is aligned with zone detector 98a. Opening 105b is aligned with zone detector 98b. Shield 92 further includes upstanding walls 106 for preventing stray radiation from entering zone detectors 98a, 98b, 100a, 100b. Upstanding walls 106 extend toward cover 64 and are positioned adjacent the reflecting and refracting elements of the optics assembly 90 when reflectometer 86 is fully assembled.

Optics assembly 90 is configured to direct the light emitted by the light sources 95, 97 to the assay strips 114 and 116. In some embodiments, optics assembly 90 is a generally planar support having at least a top face 108 and a bottom face 110. The bottom face 110 is configured to receive illumination or light emitted from the light sources 95, 97. Optics assembly 90 then directs the illumination to one or more sampling areas or reaction zones 112 on the first and second assay strips 114, 116. The top face 108 of the optics assembly 90 is also configured to transmit the diffusely reflected optical radiation returning from the sampling areas or reaction zones 112 to one or more of the zone detectors 98a, 98b, 100a, 100b.

The first and second strip assays 114, 116 may be mounted on the top face 108 of optics assembly 90 to securely hold the assay strips 114, 116 in place. Alternatively, the first and second assay strips 114 and 116 may be mounted on strip carrier, which are in turn mounted on the top face 108 of optics assembly 90.

Meter 60 further includes a power source, such as batteries, for providing power to PCB 88 and a display unit 272 coupled to cover 64. Display unit 272 may be a liquid crystal display (LCD) and is adapted for displaying assay result information. In some embodiments, display unit 272 includes a first screen 270 for displaying a numerical output corresponding, for example, to the amount of analyte detected by the reflectometer 86 and a second screen 274 for indicating the identity of the assay result by pointing to the appropriate marking or indicia on the exterior surface 68 of cover 64.

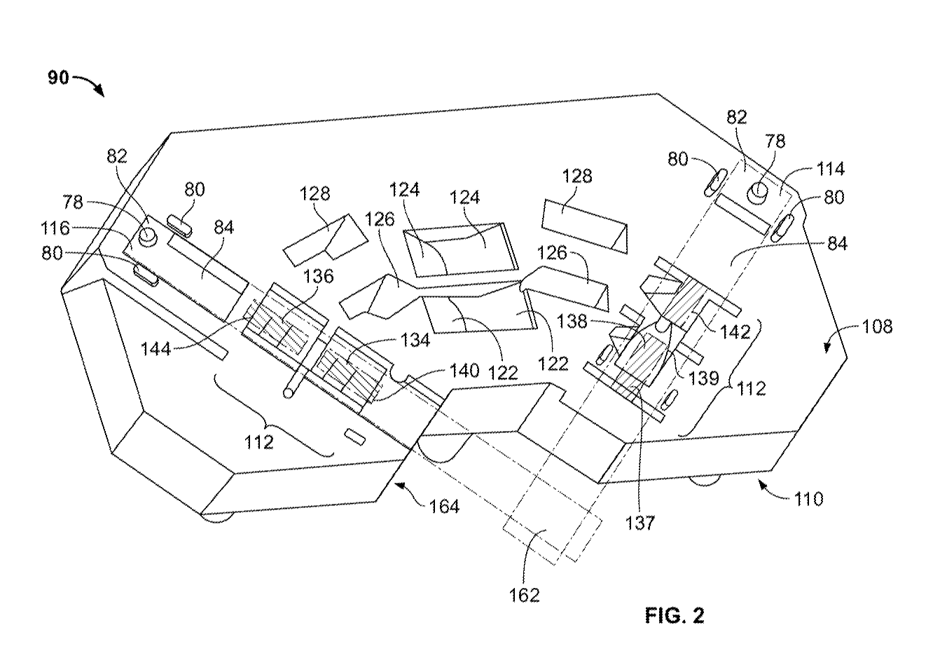

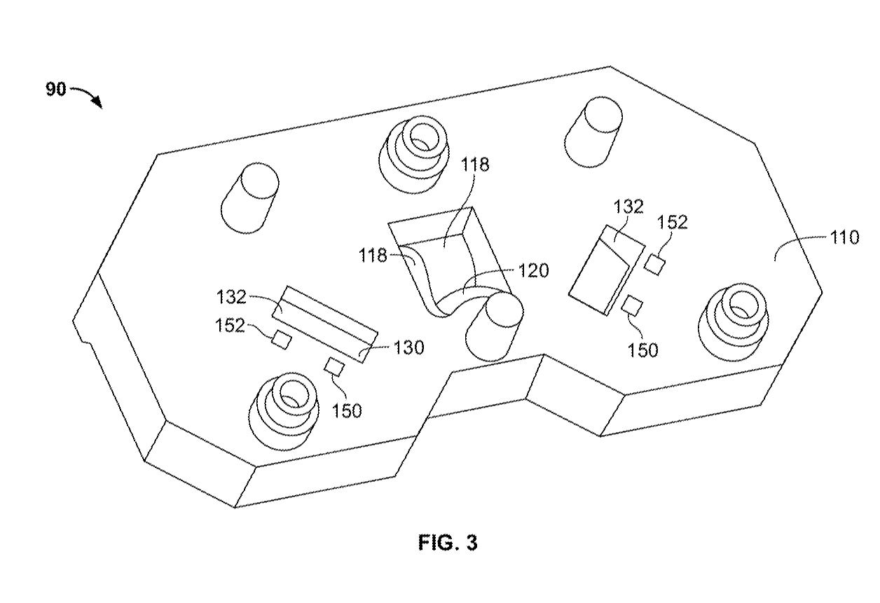

FIGS. 2 and 3 depict the top face 108 and bottom face 110 of optics assembly 90, respectively. As discussed above, optics assembly 90 is configured to transmit light or illumination emanating from light sources 95, 97 toward the sampling areas or reaction zones 112 on the first and second assay strips 114, 116 shown in phantom. To direct the light to the sampling areas or zones 112, optics assembly 90 includes a first pair of reflecting elements 122 and 124 positioned at a central portion of top face 108, a second pair of reflecting elements 126 and 128 adjacent to the first and second assay strips 114, 116 on the top face 108 and a third pair of reflecting elements 130 and 132 adjacent the first and assay strips 114, 116 on the bottom face 110. In addition, optics assembly 90 transmits the optical radiation diffusely reflected from the sampling areas 112 on the first and second assay strips 114 and 116 to one or more zone detectors 98a, 98b, 100a, 100b. One or more of the reflecting elements 122, 124, 126, 128, 130, and 132 may be total internal reflection (TIR) surfaces.

The top surface 108 of optics assembly 90 includes two indentations 84 each dimensioned for receiving one assay strip 114 or 116. Indentations 84 are aligned on top face 108 so that they position assay strips 114, 116 directly over the zone detectors 98a, 98b, 100a, 100b. Optics assembly 90 may also include walls 80 and pins 78 for securing the assay strips 114, 116 in the indentations 84.

Optics assembly 90 further includes a first pair of refracting elements 134 and a second pair of refracting elements 136. Each of the refracting elements 134, 136 is configured to spread an illumination channel or path in a predetermined shape across sampling areas 112. Specifically, the first refracting elements 134 are positioned so that they spread the illumination across first detection zones 138 and 140 on assay strips 114, 116, whereas the second refracting elements 136 are positioned so that they spread the illumination across second detection zones 142 and 144 on assay strips 114, 116. First detection zones 138 and 140 may be general chemical assay zones, while second detection zones 142 and 144 may be specific binding assay zones, or vice-versa. Thus, the chemical assay zone and the specific binding zone may be located on the same assay strip 114 or 116.

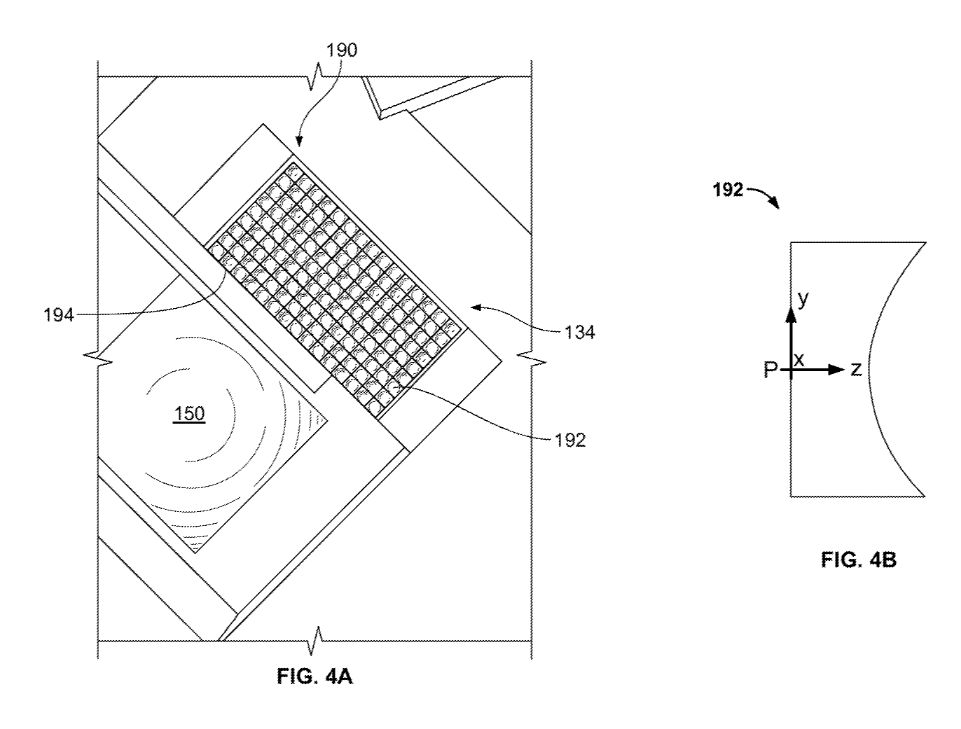

With reference to FIGS. 4A and 4B, any of the first or second pair of refracting elements 134, 136 may be composed of an array of microlenses or a lenslet array 190. The lenslet array 190 may extend within 100 μm of edge 194 and may include individual lenses 192. In some embodiments, the individual lenses 192 may be arranged in 9 rows of 15 individual lenses for a total of 135 lenses. Other embodiments may have more than 10 lenses, more than 100 lenses, or an array of lenses ranging between 10-250, although the number of lenses is not limited by the disclosure herein. Regardless of its specific arrangement, the individual lenses 192 of lenslet array 190 are spaced uniformly from one another, thereby providing uniform illumination of sampling areas 112. Uniform illumination is desirable because it offers the best means for integrating out the effects of non-uniform color development on the sampling areas 112. Therefore, uniform illumination yields a more consistent result from strip to strip than the results obtained with conventional analyte meters, which only provide non-uniform illumination of the sampling areas. Overall, the lenslet array 190 may have an area of about 2.4 mm by 1.5 mm. In some embodiments, the surface of each individual lens 192 may have a conical shape, a radius of curvature of about 100 μm, a conic constant (k) of −1, and maximum sag of about 56.25 μm. In some embodiments, each individual lens 192 has sag of about 28 μm. Each individual lens 192 may include an aperture measuring about 150 μm by 150 μm. The pitch of lenslet array 190 may be about 155 μm. The apex of the each lenslet 192 lies within 10 μm from the flat panel surface on which the lenslet is placed.

With reference again to FIG. 3, the bottom face 110 of optics assembly 90 includes a pair of refracting elements 118, 120 for partially collimating the light emitted from light sources 95, 97. Stray illumination emitted from light sources 95, 97 is directed to reference detector 96. (See FIG. 1). Each refracting element 118, 120 is configured to split light emitted into two channels or optical paths for a total of two pairs of optical paths or four individual channels of illumination. Refracting elements 118, 120 can also direct these optical paths to reflecting elements 122 and 124 (FIG. 2).

As seen in FIG. 3, optics assembly 90 includes a pair of refracting elements 150 and 152 adapted to partially collimate the diffused optical radiation from the assay strips 114 and 116 and direct it to the zone detectors 98a, 98b, 100a, 100b. Each zone detectors 98a, 98b, 100a, 100b is optically associated with a single refracting element 150 or 152 and a single detection zone 138, 140, 142, or 144. Refracting element 150 (or 152) may be any suitable lens or lens system, such as an anamorphic lens system, capable of imaging the detection zone 138 (or 140) onto detector 100a or 98a (or 100b or 98b). Moreover, refracting elements 150, 152 may be wholly or partly made of polystyrene or any other suitable material.

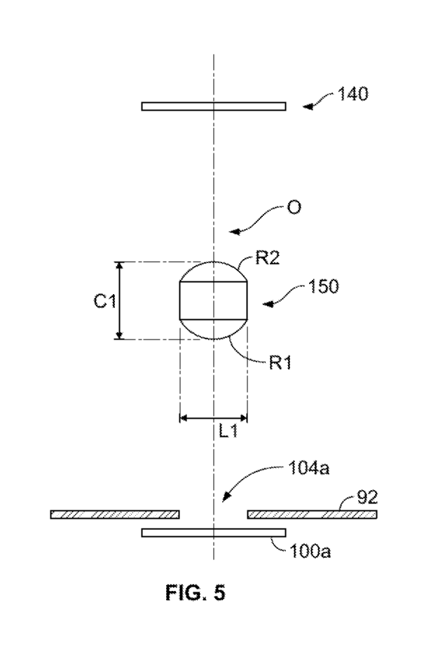

FIG. 5 shows an exemplary optical detection path, which may be representative of all optical detection paths in meter 60. As discussed above, each refracting element 150 or 152 shares an optical detection path only with a single detection zone and a single zone detector. For instance, FIG. 5 illustrates that refracting element 150 shares an optical detection path O only with a single detection zone 140 and a single zone detector 100a. In other words, a single detection zone 140 (or any other detection zone) is associated with a single zone detector 100a, a single aperture or opening 104a of shield 92, and a single refracting element 150. Detector 100a and opening 104a of shield 92 are oriented substantially orthogonally to the optical axis O of the refracting element 150. Because the zone detector 100a is substantially normal or perpendicular to the optical axis O of refracting element 150, the signal generated by the zone detector 100a will be higher than in conventional designs in which the zone detectors are oriented at an oblique angle with respect to the optical axis of the refracting element. In addition, since the diffused optical radiation passing through opening 104a of shield 92 is being imaged on the zone detector 100a, the tolerance in placing the zone detector 100a on the PCB 88 is virtually irrelevant during manufacturing, so long as the active area of zone detector 100a is larger than the mechanical tolerances in locating the opening 104a with respect to the refracting element 150. As a result, the output signal generated by zone detector 100a will be more consistent from monitor to monitor in comparison with conventional designs. Since the image of opening 100a is smaller than the detection zone 140, the presently disclosed design allows some tolerance in locating the detection zone 140 without impacting the optical radiation reflected from detection zone 140.

In some embodiments, the zone detector 100a (or any other zone detector) may have an active area measuring at least about 1.2 by 1.6 mm. Opening 104a (or 104b) of shield 92 may measure about 0.5 by 0.9 mm. The magnification of refracting element 150 (or 152) may be 2× when refracting element 150 (or 152) has a first surface radius R1 of about 2.9032 mm and a second surface radius R2 of about 1.0256 mm and a conic constant (k) of −1.0 on the second surface. This specific embodiment yields a detector field of view on the zone detector 100a of about 1.0 by 1.8 mm. Alternatively, the first surface radius R1 of refracting element 150 (or 152) may be 1.2 mm and the second surface radius R2 may be 1.4 mm. Refracting element 152 may have a cross-sectional area of about 1.8 mm by 2.0 mm and a width L1 of about 1.64 mm. Because the field of view on the zone detector 100a is smaller (e.g., 1.0×1.8 mm) than the zone detector 100a itself (e.g., 1.5×2.7 mm), the detection zone 140 may be moved to a certain degree relative to the optics axis O without impacting the signal measured by the zone detector 100a. Since the active area of the zone detector 100a is larger than the aperture 102, the tolerances for placing the detector 100a on the PCB 88 may be higher without impacting the signal measured by the zone detector 100a. Moreover, the field of view of the zone detector 100a is fully contained within the area of the detection zone 140 and, consequently, yields more precise results from strip-to-strip because zone detector 100a is less likely to receive noise (or stray optical radiation) from other detection zones. As discussed above, all the refracting elements, zone detectors, and detection zones of optics assembly 90 may also have the features and measurements described above.

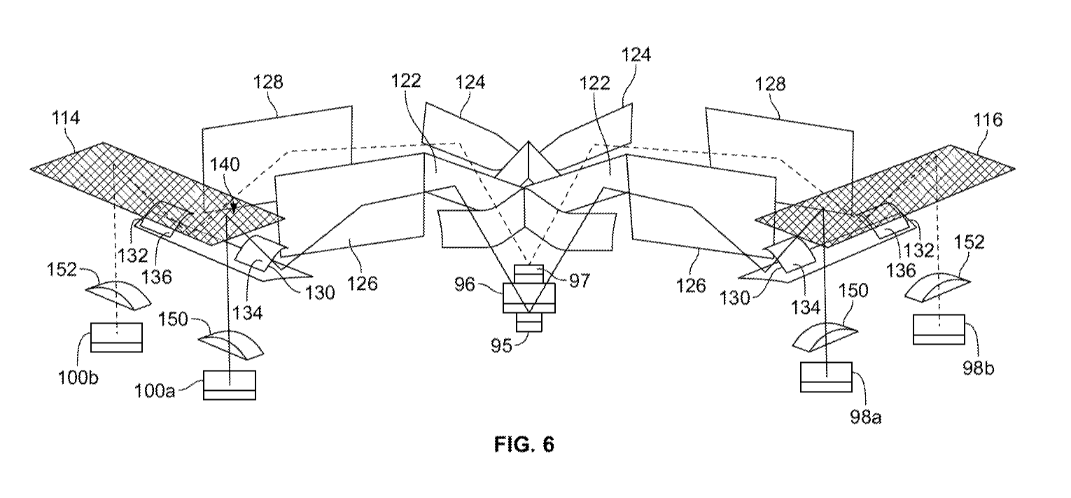

In operation, analyte meter 60 quantitatively measures HbA1c or any other preselected analyte in a fluid sample 72. In doing so, optics assembly 90 directs light emanating from light sources 95, 97 as schematically illustrated in FIG. 6. With reference to FIG. 1, first, a sample 72 containing one or more selected analytes is introduced into sample receiving device or receptor 74 through inlet port 66 of cover 64. Sample receiving device or receptor 74 receives at least a portion of sample 72 and distributes the received sample 72 between the two assay strips 114, 116. The operation of analyte meter 60 may commence automatically by sensing the introduction of sample 72 with any suitable sensing mechanism, which in turn generates a signal to activate the analyte meter 60. U.S. Pat. No. 5,837,546, the entire disclosure of which is hereby incorporate by reference, describes a sensing mechanism for sensing introduction of a sample into housing 62 of analyte meter 60.

Upon activation of analyte meter 60, light sources 95, 97 emit optical radiation or light toward optics assembly 90. In an embodiment where analyte meter 60 measures hemoglobin A1c, at least one of the light sources 95 or 97 emits green light and the other light source emits red light. The green-emitting light source 95 or 97 emits optical radiation having a wavelength substantially similar to the maximum absorption band of Hb. In some embodiments, the green-emitting light source is adapted to emit optical radiation with a wavelength ranging between 525-535 nm. In one embodiment, a wavelength of 530 nm produces optimum results. This is in contrast to conventional analyte meters which include a light source that emits green light at a much higher wavelength of about 565 nm.

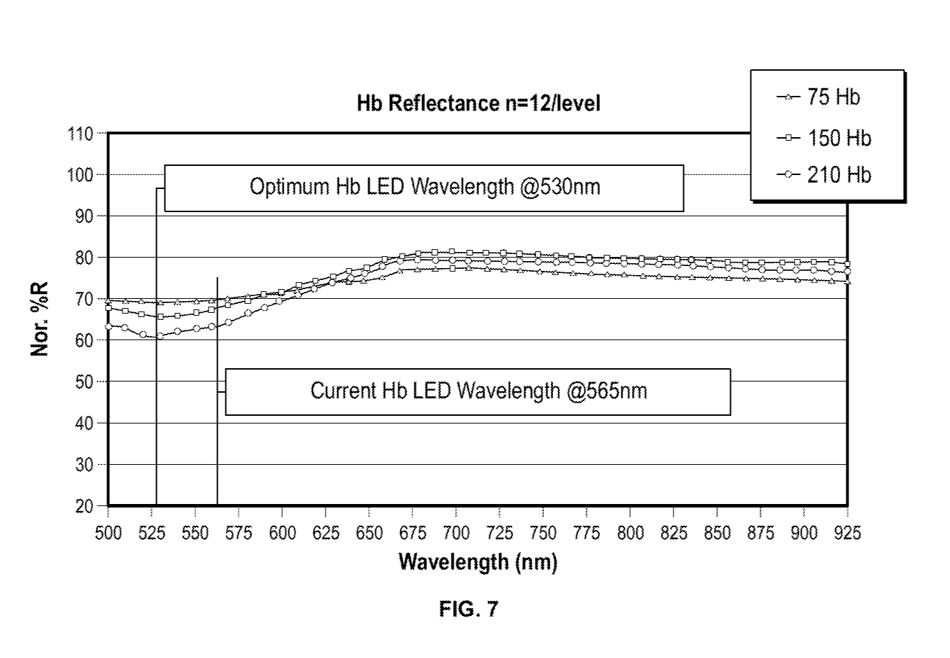

FIG. 7 shows the reflectance curves for the Hb sample detection zones (138, 140, 142 or 144). These reflectance curves for different Hb concentrations are the plot of the reflectivity (measured in percent reflectance (% R)) as a function of wavelength (measured in nanometers). As seen in FIG. 7, it was discovered that in order to maximize the resolution of the measurement (i.e., change in % R/change in analyte concentration), it is desirable to choose a green light source centered in a range between 525-535 nm, instead of the conventional 565 nm. Selecting a green light source centered at 530 nm (or within the range of 525-535) provides for optimal results, as evidenced by the fact that the greatest vertical separation between the lowest and highest concentrations of Hb occurs at a wavelength of 530 nm. In other words, adjusting the green light source to about 530 nm (or within the range of 525-535 nm) increases the dynamic range of the reflectance measurement for measuring Hb. This, in turn, provide for more accurate test results, as compared to conventional analyte meters that have a green light source emitting light at 565 nm. The presently disclosed analyte meter 60 therefore increases the dynamic range of the reflectance measurement from 6.7% to 8.4%.

Referring again to FIG. 6, light sources 95 and 97 emit light. Each refracting element 118, 120 (FIG. 3) splits light emitted from light sources 95, 97 into two channels or optical paths for a total of two pairs of optical paths or four individual channels of illumination. The first pair of reflecting elements 122 and 124 directs the illumination to the second pair of reflecting elements 126 and 128. Then, the second pair of reflecting elements 126 and 128 direct the illumination to the third pair of reflecting elements 130 and 132. As discussed above, at least one of the reflecting elements 122, 124, 126, 128, 130, and 132 may be total reflective surfaces (TIR). The illumination is then passed through pairs of refracting elements 134 and 136, which spread the illumination for each channel in a predetermined shape across sampling areas 112 of first and second assay strips 114, 116. Specifically, the pair of refracting elements 134 spread the illumination across first detection zones 138 and 140 on assay strips 114 and 116, respectively. The pair of refracting elements 136 spread the illumination across second detection zones 142 and 144 on assay strips 114 and 116, respectively.

Diffused optical radiation is reflected downward by the first detection zones 138 and 140 and second detection zones 142, 144. Pairs of refracting elements 150 and 152 direct diffused optical radiation to zone detectors 98a, 98b, 100a, 100b. Specifically, zone detector 98a receives the diffused optical radiation from the first detection zone 138 on the first assay strip 114. Detector 98b receives the diffused optical radiation from the second detection zone 142 on the first assay strip 114. Zone detector 100a receives the diffused optical radiation from the first detection zone 140 on the second assay strip 116. Zone detector 100b receives the diffused optical radiation from the second detection zone 144 on the second assay strip 116.

Zone detectors 98a, 98b, 100a, and 100b detect and measure the reaction occurring on each assay strip 114, 116. For example, optics assembly 90 can be used to detect the blood/analyte reaction occurring on strip 114 which correlates to hemoglobin A1 (HbA1c) concentration in the blood sample. In some embodiments, zone detectors 98a, 98b, 100a, and 100b are photodetectors that measure reflectance from assay strips 114 and 116 and then generate an electrical signal, which correlates with the reflectance measurement. The concentration of HbA1c or any other analyte is determined from the reflectance in the detection zones. A mathematical algorithm is used to define the concentration of the analyte as a function of the reflectance in the detection zones. U.S. Patent Application Publication No. 2005/0227370, the entire content of which is herein incorporate by reference, describes algorithms for defining the concentration of an analyte as a function of the reflectance in the detection zones. However, any known method of calculating the concentration of an analyte may be utilized. The processor mounted on the PCB 88 analyzes the results of the optical detection and then visually displays the results on display unit 272.

The below paragraphs identify certain features of some of the embodiments disclosed herein.

1. An analyte meter for an analyte test strip, comprising:

a light source configured to emit a light having a wavelength substantially similar to the maximum absorption band of Hb;

an optics assembly configured to direct the light emitted by the light source to a test strip; and

a photodetector configured to quantitatively detect light emanating from the test strip and to generate a signal correlating to an analyte concentration in the test strip.

2. The analyte meter according to paragraph 1, wherein the light source is configured to emit a green light having a wavelength of 530 nm.

3. An analyte meter, comprising:

a first light source configured to emit a first light;

a second light source configured to emit a second light;

an assay strip including first and second reaction zones, the first reaction zone being adapted for receiving a fluid sample containing a first analyte and a second analyte and including a first reagent capable of inducing an optical change in the fluid sample when reacted with the first analyte, the second reaction zone being adapted for receiving the fluid sample and including a second reagent capable of inducing an optical change in the fluid sample when reacted with the second analyte;

an optics assembly configured to direct the first light to the first reaction zone and the second light to the second reaction zone;

a first photodetector positioned to detect only optical radiation reflected from the first reaction zone and to generate a first signal indicative of an amount of analyte in the fluid sample positioned in the first reaction zone;

a second photodetector positioned to detect only optical radiation reflected from the second reaction zone and to generate a second signal indicative of an amount of analyte in the fluid sample positioned in the second reaction zone.

4. The analyte meter of paragraph 3, wherein the first analyte and the second analyte are different analytes.

5. An analyte meter for detecting an analyte concentration in a test strip having a reaction zone adapted for receiving a fluid sample containing an analyte and including a reagent capable of inducing an optical change in the fluid sample when reacted with the analyte, comprising:

a light source configured to emit a light along an illumination path;

an optics assembly configured to direct the light emitted by the light source to the reaction zone of the test strip, the optics assembly including an array of lenses positioned along the illumination path; and

a photodetector configured to quantitatively detect light reflected from the reaction zone and to generate a signal correlating to an amount of analyte in the fluid sample.

6. The analyte meter according to paragraph 5, wherein the lenses in the array of lenses are uniformly spaced apart from one another.

7. An optics assembly for an analyte meter system, comprising:

a receiving portion adapted for receiving at least one test strip, the at least one test strip having a reaction zone adapted for receiving a fluid sample containing an analyte and including a reagent capable of inducing an optical change in the fluid sample when reacted with the analyte;

a light source configured to emit a green light having a wavelength substantially similar to the maximum absorption band of Hb;

at least one reflecting element configured to direct the light emitted by the light source to the reaction zone of the test strip placed in said receiving portion;

a photodetector configured to quantitatively detect light emanating from the reaction zone of the test strip and to generate a signal correlating to an amount of analyte in the fluid sample.

It will be appreciated that various features set forth in the embodiments discussed herein can be combined in different ways then presented herein. It will also be appreciated that the features described in connection with individual embodiments may be shared with other embodiments discussed herein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An analyte meter for an analyte test strip, comprising:
a housing;
a light source in the housing configured to emit a light having a wavelength substantially similar to the maximum absorption band of Hb;
an optics assembly configured to direct the light emitted by the light source to a test strip; and
a photodetector configured to quantitatively detect light emanating from the test strip and to generate a signal correlating to an analyte concentration in the test strip.

2. The analyte meter according to claim 1, wherein the light source is configured to emit a green light having a wavelength ranging between 525-535 nm.

3. The analyte meter according to claim 2, wherein the green light has a wavelength of 530 nm.

4. The analyte meter according to claim 1, further comprising a refracting element for directing the light to the test strip, the refracting element comprising a plurality of lenses.

5. The analyte meter according to claim 4, wherein the plurality of lenses are arranged in an array.

6. The analyte meter according to claim 5, wherein prior to the light reaching the test strip, the array of the plurality of lenses is the last surface area through which light travels.

7. The analyte meter according to claim 5, wherein the plurality of lenses in the array of lenses are uniformly spaced apart from one another.

8. The analyte meter according to claim 5, wherein there are more than 10 lenses arranged in the array of lenses.

9. The analyte meter according to claim 8, wherein the number of lenses in the array ranges between 10 and 250.

10. The analyte meter according to claim 1, wherein there are more than 100 lenses in the array.

11. The analyte meter according to claim 5, wherein a surface of at least one lens in the array of lenses has a radius of curvature of about 100 μm, a conic constant (k) of −1, and maximum sag of about 56.25 μm.

12. The analyte meter according to claim 5, where a pitch of the array of lenses is about 155 μm.

13. An analyte meter, comprising:
a housing;
a first light source in the housing configured to emit a first light;
a second light source configured to emit a second light;
an assay strip including first and second reaction zones, the first reaction zone being adapted for receiving a fluid sample containing a first analyte and a second analyte and including a first reagent capable of inducing an optical change in the fluid sample when reacted with the first analyte, the second reaction zone being adapted for receiving the fluid sample and including a second reagent capable of inducing an optical change in the fluid sample when reacted with the second analyte;
an optics assembly configured to direct the first light to the first reaction zone and the second light to the second reaction zone;
a first photodetector positioned to detect only optical radiation reflected from the first reaction zone and to generate a first signal indicative of an amount of analyte in the fluid sample positioned in the first reaction zone;
a second photodetector positioned to detect only optical radiation reflected from the second reaction zone and to generate a second signal indicative of an amount of analyte in the fluid sample positioned in the second reaction zone.

14. The analyte meter of claim 13, wherein the first analyte and the second analyte are different analytes.

15. The analyte meter of claim 13, wherein the optics assembly further comprises a first refracting element positioned between the first reaction zone and the first photodetector, the first refracting element having an optical axis extending through the first refractor in a direction between the first reaction zone and the first photodetector, the first photodetector extending in a direction perpendicular to the optical axis.

16. The analyte meter of claim 15, wherein the optics assembly further comprises a second refracting element positioned between the second reaction zone and the second photodetector, the second refracting element having a second optical axis extending through the second refracting element in a direction between the second reaction zone and the second photodetector, the second photodetector extending in a direction perpendicular to the optical axis.

17. The analyte meter of claim 13, wherein the optics assembly further comprises a plurality of reflecting elements for directing the light emanating from the first light source to the first reaction zone.

18. The analyte meter of claim 17, wherein the first light source is aligned with at least one reflecting element.

19. An analyte meter for detecting an analyte concentration in a test strip having a reaction zone adapted for receiving a fluid sample containing an analyte and including a reagent capable of inducing an optical change in the fluid sample when reacted with the analyte, comprising:
a light source configured to emit a light along an illumination path;
an optics assembly configured to direct the light emitted by the light source to the reaction zone of the test strip, the optics assembly including an array of microlenses positioned along the illumination path; and
a photodetector configured to quantitatively detect light reflected from the reaction zone and to generate a signal correlating to an amount of analyte in the fluid sample.

20. The analyte meter according to claim 19, wherein the microlenses in the array of microlenses are uniformly spaced apart from one another.

21. An optics assembly for an analyte meter system, comprising:
a receiving portion adapted for receiving at least one test strip, the at least one test strip having a reaction zone adapted for receiving a fluid sample containing an analyte and including a reagent capable of inducing an optical change in the fluid sample when reacted with the analyte;
a light source configured to emit a green light having a wavelength substantially similar to the maximum absorption band of Hb;
at least one reflecting element configured to direct the light emitted by the light source to the reaction zone of the test strip placed in said receiving portion;
a photodetector configured to quantitatively detect light emanating from the reaction zone of the test strip and to generate a signal correlating to an amount of analyte in the fluid sample.

22. The optics assembly of claim 21, wherein the green light has a wavelength ranging between 525-535 nm.
